(19) United States
(12) Patent Application Publication
(10) Pub. No.: US 2018/0361133 A1
(43) Pub. Date: Dec. 20, 2018

(54) INTRAVENOUS LINE PORT COVER

(71) Applicant: Jacklyn Herron, Tinton Falls, NJ (US)

(72) Inventor: Jacklyn Herron, Tinton Falls, NJ (US)

(21) Appl. No.: 16/009,834

(22) Filed: Jun. 15, 2018

Related U.S. Application Data
(60) Provisional application No. 62/520,296, filed on Jun. 15, 2017.

(51) Int. Cl. A61M 39/02; A61F 13/38; A61M 25/00

(52) U.S. Cl. CPC A61M 39/0247; A61F 13/38; A61M 2209/06; A61M 2039/0258; A61M 2039/0288; A61M 25/0097

(57) ABSTRACT

An intravenous line port cover. The intravenous line port cover is formed by the connection of a first member to a second member. The first member and the second member form a cavity that can hold an intravenous line port. The housing has a pair of apertures that receive the intravenous lines leading into and out of the intravenous line port. Each aperture has a washer that forms a seal around the intravenous lines that prevents contaminants from reaching the intravenous line port.

### CROSS REFERENCE TO RELATED APPLICATIONS

This application claims the benefit of U.S. Provisional Application No. 62/520,296 filed on Jun. 15, 2017. The above identified patent application is herein incorporated by reference in its entirety to provide continuity of disclosure.

### BACKGROUND OF THE INVENTION

The present invention relates to an intravenous line port cover. The port of an intravenous line is a weak point in the intravenous line. Because of the presence of this weak point, failure to protect the line port cover may result in contaminants entering a patient, potentially causing an infection.

Currently available device purport to cover intravenous line ports. However, these devices fail to provide a sufficient microbial seal. Therefore, there is a need for an intravenous line port cover that provides a proper microbial seal. By providing a proper microbial seal, medical procedures involving intravenous lines can be made safer and more sanitary.

### SUMMARY OF THE INVENTION

In view of the foregoing disadvantages inherent in the known types of intravenous line port covers now present in the known art, the present invention provides an intravenous line port cover wherein the same can be utilized for providing convenience for the user when covering a port of an intravenous line.

The present system comprises a first member moveably attached to a second member. The first member corresponds to the second member forming a housing therebetween. The housing defines a first aperture and a second aperture. Each aperture has a washer interacting therewith, such as to form a seal around an intravenous line.

It is another object of the present invention to provide a first member and second member that are each defined by a prolate hemispheroid shape such as to provide a housing dimensioned to contain an intravenous line port.

Yet another object of the present invention is to provide a pocket disposed on an interior surface of either the first member or the second member, such that an object can be stored in the pocket.

A further object of the present invention is to provide a sanitary swab disposed in the pocket, such that the sanitary swab can be used to sanitize the intravenous port cover.

Yet another object of the present invention is to provide an antiseptic substance in the pocket, such as chlorhexidine, such that the contents of the pocket can be sanitized.

An additional object of the present invention is to provide a plurality of washers that are removable from each aperture, such as to provide convenience to a user when creating a seal with the intravenous line.

Yet another object of the present invention is to provide a washer that is integrated upon each aperture such that a microbial seal is formed with the intravenous line.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the intravenous line port cover in an open position.

FIG. 2 shows a perspective view of an embodiment of the intravenous line port cover in a closed position.

FIG. 3 shows a perspective view of an embodiment of the intravenous line port cover in an open position with a intravenous line port disposed therein.

FIG. 4 shows a close-up view of a washer of an embodiment of the intravenous line port cover in use.

FIG. 5 shows a perspective view of an alternate embodiment of the intravenous line port cover in use.

### DETAILED DESCRIPTION OF THE INVENTION

Reference is made herein to the attached drawings. Like reference numerals are used throughout the drawings to depict like or similar elements of the intravenous line port cover. The figures are intended for representative purposes only and should not be considered to be limiting in any respect.

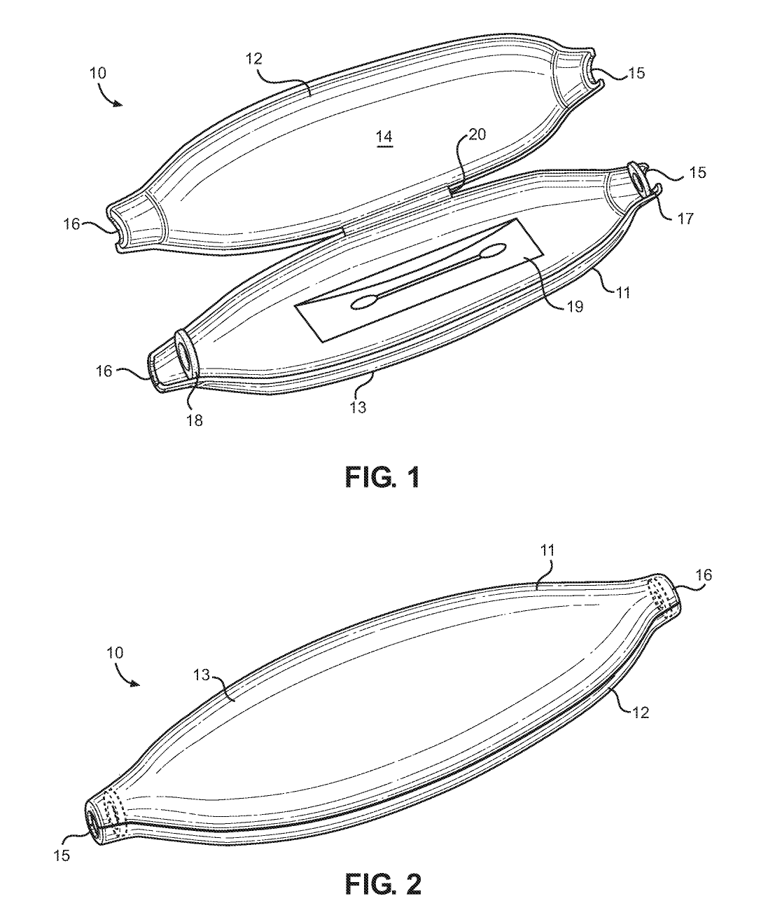

Referring now to FIG. 1, there is shown a perspective view of an embodiment of the intravenous line port cover in an open position. The intravenous line port cover 10 comprises a housing 13. The housing 13 comprises a first member 11 movably attached to a second member 12. In the shown embodiment, the first member 11 and the second member 12 are of a prolate hemispheroid shape. Furthermore, in the shown embodiment, the first member 11 is dimensionally identical to the second member.

The first member 11 and the second member 12 are concavo-convexly arranged, such that the housing 13 defines a chamber 14 therein. The first member 11 and the second member 12 are separable along a central longitudinal axis. The chamber 14 is dimensioned to receive and secure an intravenous line port therein. In one embodiment, the first member 11 couples to the second member 12 via a fastener.

The housing 13 is selectively movable between a closed position and an open position. In the closed position. The first member 11 and the second member 12 are joined to encompass an intravenous line port/assembly in the housing 13. In the closed position, an aperture 15, 16 is formed cooperatively at an end thereof.

In one embodiment, a washer 17, 18 is disposed on each aperture 15, 16. In one embodiment, each washer 17,18 is integrated upon each of a first aperture 15 and a second aperture 16. In another embodiment, each washer 17, 18 is removable from each of the first aperture 15 and the second aperture 16.

The washer 17, 18 is disposed adjacent to the aperture, the washer 17, 18 is adapted to form a seal annularly about the intravenous line port/assembly and between an interior of the housing 13 and an exterior of the housing 13. Wherein in the closed position, the washer 17, 18 is disposed circumfluent to an inside surface of the housing 13 and thereby prevents fluid communication thereacross.

The first member 11 is movable attached to the second member 12. In the shown embodiment, the first member 11 is attached to the second member 12 by a hinge 20. The hinge 20 allows the first member 11 and the second member 12 to be moved relative to each other such that the intravenous line port cover 10 can be moved from an open position to a closed position (see FIG. 2).

In the shown embodiment, a pocket 19 is disposed on an interior surface of the first member 11. In an alternate embodiment, the pocket 19 is disposed on an interior surface of the second member 12. The pocket 19 is dimensioned to receive an object therein. In the shown embodiment, a sanitary swab 21 is disposed in the pocket 19. The sanitary swab 21 may be suspended in an antiseptic substance, such as chlorhexidine.

Referring now to FIG. 2, there is shown a perspective view of an embodiment of the intravenous line port cover in a closed position. The closed position of the intravenous line port cover 10 is defined where the first member 11 and the second member 12 are joined to enclose an intravenous line port within the housing 13. Furthermore, in the closed position, the intravenous line port will be enclosed and secured therein, and the intravenous line of the intravenous line port extend through the aperture 15, 16. The washers 17,18 form microbial seals, such as to prevent contaminants from coming into contact with the intravenous line port.

In the shown embodiment, the intravenous line port cover 10 is frictionally held in the closed position when placed therein by a user. In another embodiment, a locking mechanism is utilized to hold the intravenous line port cover in a closed position. The locking mechanism is configured to secure the intravenous line port cover 10 in a closed position. For example, in one embodiment, the locking mechanism comprises a latch configured to be insertable into an indentation.

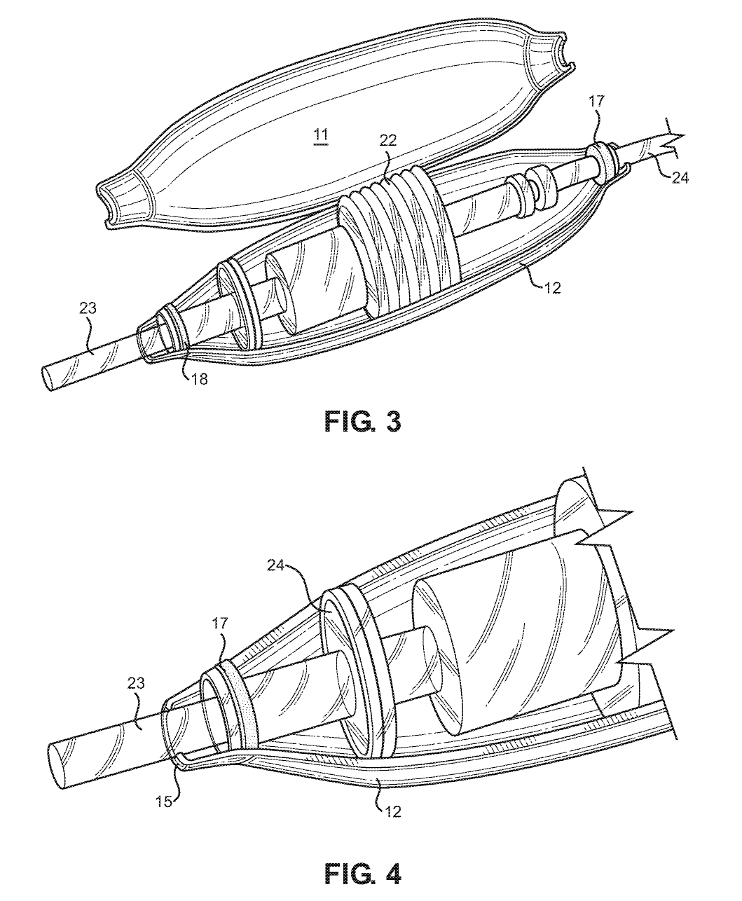

Referring now to FIG. 3, there is shown a perspective view of an embodiment of the intravenous line port cover in an open position with an intravenous line port disposed therein. The first member 11 and the second member 12 are each dimensioned such that the housing formed therebetween is sized to contain an intravenous line port 22. Each of the first aperture 15 and the second aperture 16 is dimensioned to receive an intravenous line 23, 24 therein. The washers 17,18 are configured to form a seal around the intravenous lines 23, 24 that pass through the first aperture 15 and the second aperture 16.

In the shown embodiment, the apertures 17, 18 are axially aligned along a center line. The housing 13, when in the closed position has a circular cross section taken along a midpoint between the ends. Additionally, the housing 13 tapers from a medial portion to each end.

Referring now to FIG. 4, there is shown a close-up view of a washer of an embodiment of the intravenous line port cover in use. In the shown embodiment, the washer 17 is attached to the second member 12 at the first aperture 15. The washer 17 is dimensioned to form a seal around an intravenous line 23. In one embodiment, the washer 17 is removable from the second member 23. Removability of the washer 17 allows a user to place the washer on the intravenous line port 22 before the intravenous line 24 is attached thereto. Thus, the seal can be formed at a user-specified location. In one embodiment, the washer 17 is flexible. In another embodiment, the washer 17 is made of silicone.

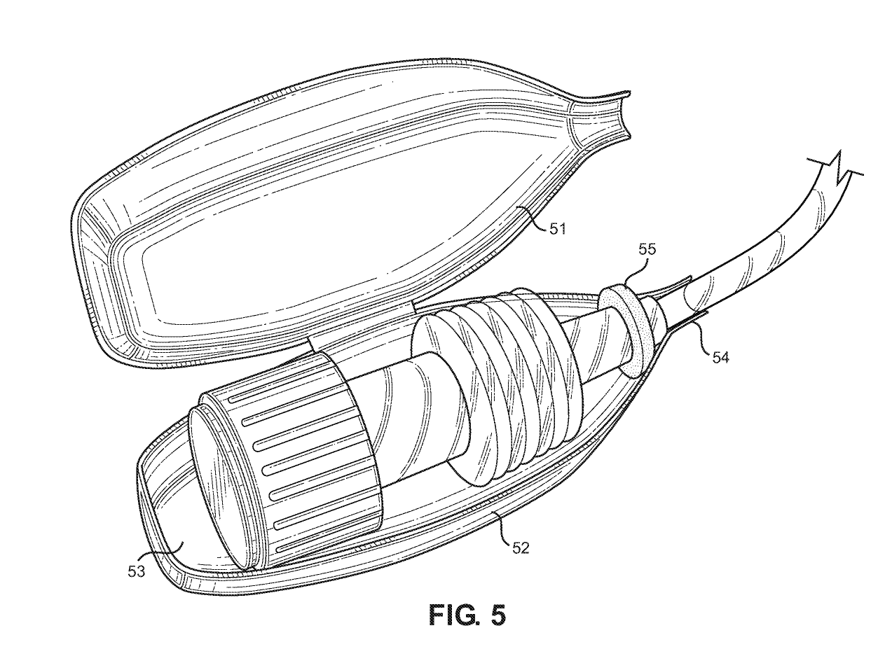

Referring now to FIG. 5, there is shown a perspective view of an alternate embodiment of the intravenous line port cover in use. In the shown embodiment, the first member 51 is movably attached to the second member 52. The first member 51 and the second member 52 form a housing having a cavity 53. The housing defines a singular aperture 54. The singular aperture 54 includes a washer 55 corresponding thereto. The washer 55 is dimensioned to form a seal around an intravenous line.

In the shown embodiment, the singular aperture 54 is disposed centrally on an end of the housing. The housing has a cylindrical shape in the closed position. Additionally, the housing tapers from a medial portion to the end having the singular aperture 54.

In one embodiment, a pocket is disposed on an interior surface of either the first member 51 or the second member 52. In a further embodiment, a sanitary swab may be disposed in the pocket. In another embodiment, the sanitary swab is disposed in an antiseptic substance, such as chlorhexidine.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An intravenous line port cover, comprising:
a housing comprising a first member movably attached to a second member, the housing selectively movable between a closed position and an open position;
wherein in the closed position the first member and the second member are joined to encompass an intravenous line port/assembly therein and cooperatively form an aperture at an end thereof;
the aperture configured to receive an intravenous line of the intravenous line port/assembly therethrough; and
a washer disposed adjacent the aperture, the washer adapted to form a seal annularly about the intravenous line and between an interior of the housing and an exterior of the housing.

2) The intravenous line port cover of claim 1, wherein the first member and the second member each is defined by a prolate hemispheroid shape.

3) The intravenous line port cover of claim 1, wherein a pocket is disposed on an interior surface of at least one of the first member and the second member.

4) The intravenous line port cover of claim 3, wherein a sanitary swab is disposed in the pocket.

5) The intravenous line port cover of claim 4, wherein the sanitary swab is disposed in an antiseptic substance.

6) The intravenous line port cover of claim 5, wherein the antiseptic substance is chlorhexidine.

7) The intravenous line port cover of claim 1, wherein each washer is integrated upon each aperture.

8) The intravenous line port cover of claim 1, wherein each washer is removable from each aperture.

9) The intravenous line port cover of claim 1, wherein each washer is flexible.

10) The intravenous line port cover of claim 1, wherein each washer is made of silicone.

11) The intravenous line port cover of claim 1, wherein the housing tapers from a medial portion to each end.

12) The intravenous line port cover of claim 1, wherein the first member is dimensionally identical to the second member.

13) The intravenous line port cover of claim 1, wherein the first member is affixed to the second member via a hinge.

14) The intravenous line port cover of claim 1, wherein an edge of the first member frictionally engages with an edge of the second member to form the housing.

15) The intravenous line port cover of claim 1, wherein the first member couples to the second member via a fastener.

16) The intravenous line port cover of claim 1, wherein the first member and the second member of the housing are separable along a central longitudinal axis.

17) The intravenous line port cover of claim 1, wherein the closed position the washer is disposed circumfluent to an inside surface of the housing and thereby prevents fluid communication thereacross.
